(19) United States
(12) Patent Application Publication
(10) Pub. No.: US 2011/0009604 A1
(43) Pub. Date: Jan. 13, 2011

(54) Method for Concentrating, Purifying and Removing Prion Protein

(75) Inventors: Ralph Zahn, Zurich (CH); Ahmed El Gedaily, Zurich (CH); Susanne Franitza, Zurich (CH); Nicola Franscini, Zurich (CH); Ulrich Matthey, Zurich (CH)

Correspondence Address: THE NATH LAW GROUP, 112 South West Street, Alexandria, VA, 22314, US

(73) Assignee: Alicon AG, Zurich (CH)

(21) Appl. No.: 12/083,971

(22) PCT Filed: Oct. 25, 2006
(86) PCT No.: PCT/EP06/10272

§ 371 Date: Apr. 23, 2008

(30) Foreign Application Priority Data
Oct. 28, 2005 (EP) PCT/EP2005/011565

(51) Int. Cl. C07K 1/22; C07H 1/00

(52) U.S. Cl. 530/413; 536/123.1

(57) ABSTRACT

The present invention relates to a method for concentrating and/or purifying prion PrP^{Sc} proteins by contacting prion PrP^{Sc} proteins with sepharose under conditions that allow for the specific and high affinity binding of the sepharose to the prion PrP^{Sc} proteins and removing the unbound non-prion proteins from the sepharose, as well as the same method for removing prion PrP^{Sc} proteins from body fluids by contacting body fluids with sepharose under conditions that allow for the specific and high affinity binding of the sepharose to the prion PrP^{Sc} proteins and removing the body fluid from said sepharose. In addition, the present invention is directed to a method for separating and/or enriching prion PrP^{Sc} proteins from PrP^{C} proteins by contacting prion PrP^{Sc} proteins and PrP^{C} proteins with a ligand-modified sepharose under conditions that allow for the specific and high affinity binding of the sepharose part to the prion PrP^{Sc} proteins and the binding of the ligand part of the sepharose to PrP^{C} proteins, adding a selective release agent to the sepharose-bound proteins under conditions that allow for the release of non-prion proteins and PrP^{C} proteins from the ligand part of the sepharose but not for the release of the prion PrP^{Sc} proteins, and removing the non-prion proteins and PrP^{C} from the sepharose. Another aspect of the present invention concerns the use of the before-mentioned methods for concentrating, purifying and/or removing prion PrP^{Sc} proteins.

The present invention relates to a method for concentrating and/or purifying prion PrP^{Sc} proteins by contacting prion PrP^{Sc} proteins with sepharose under conditions that allow for the specific and high affinity binding of the sepharose to the prion PrP^{Sc} proteins and removing the unbound non-prion proteins from the sepharose, as well as the same method for removing prion PrP^{Sc} proteins from body fluids by contacting body fluids with sepharose under conditions that allow for the specific and high affinity binding of the sepharose to the prion PrP^{Sc} proteins and removing the body fluid from said sepharose.

In addition, the present invention is directed to a method for separating and/or enriching prion PrP^{Sc} proteins from PrP^{C} proteins by contacting prion PrP^{Sc} proteins and PrP^{C} proteins with a ligand-modified sepharose under conditions that allow for the specific and high affinity binding of the sepharose part to the prion PrP^{Sc} proteins and the binding of the ligand part of the sepharose to PrP^{C} proteins, adding a selective release agent to the sepharose-bound proteins under conditions that allow for the release of non-prion proteins and PrP^{C} proteins from the ligand part of the sepharose but not for the release of the prion PrP^{Sc} proteins, and removing the non-prion proteins and PrP^{C} proteins from the sepharose.

Another aspect of the present invention concerns the use of the before-mentioned methods for concentrating, purifying and/or removing prion PrP^{Sc} proteins.

The Sequence Listing submitted in text format (.txt) on Sep. 24, 2010, named “29243U_Sequence_Listing.txt, (created on Sep. 16, 2010, 2 KB), is incorporated herein by reference.

### BACKGROUND OF THE INVENTION

Native prion protein, referred to as “PrP^{C}” for cellular prion protein, is widely distributed throughout nature and is particularly well conserved in mammals. The conversion of the native PrP^{C} protein to the infectious protein, referred to as “PrP^{Sc}” for scrapie prion protein or as “PrP^{res}” for proteinase K resistant prion protein, is believed to lead to the propagation of various diseases. Examples of prion-associated diseases include, for example, kuru and Creutzfeldt-Jakob disease (CJD) in humans; scrapie in sheep, bovine spongiform encephalopathy (BSE) in cattle, transmissible mink encephalopathy and wasting disease in deer and elk.

BSE is a form of mad cow disease and is transmissible to a wide variety of other mammals including humans. The human form of BSE is referred to as new variant Creutzfeldt-Jakob disease or vCJD. An estimated 40 million people in the United Kingdom ingested BSE-contaminated beef during the mid- to late 1980s. Because the incubation period for the orally transmitted disease may be 20-30 years, the true extent of this disease may not become apparent until after 2010.

In addition to the ingestion of infected beef, there is a potential for the transmission of prion-associated diseases among humans by blood transfusion. Since there are now (two) direct indications of prion transmission by blood transfusions, there is increasing concern about the security of blood products. Also, the infected prions have already been shown to be present on lymphocytes, and there is also evidence indicating that prions are present in the plasma in addition to being cell-associated. Furthermore, animals can become infected with prion-associated diseases by grazing on prion-contaminated soil or by ingesting hay that contains prion-infected hay mites.

The ability to detect and also to remove prion proteins from a sample is of profound importance in the food industry and the medical sector.

For detecting prion proteins a number of assays based on prion-specific antibodies have been developed. However, these assays require prior enrichment due to the very low concentrations of prion proteins in nature and in mammals, particularly in human blood, human or other mammalian organs for transplantation and in meat and processed foods derived from mammals.

A number of approaches for purifying prion proteins and derivatives thereof have been developed during the last decade. Affinity chromatography plays a major role as a suitable purification technique. In particular, sepharose gels have proven themselves as suitable support material for carrying ligands for affinity chromatography.

Grathwohl et al. (Arch. Virol. (1996) 141: 1863-1874) disclose the enrichment of PrP^{Sc} from mouse spleen of Scrapie-infected mice shortly after infection through immobilized metal (Cu²⁺) affinity chromatography (IMAC) employing divalent copper ion sepharose as support material. However, they found that for the diagnosis at the earliest stage of infection, extraction of PrP^{Sc} by salting out with Sarkosyl and NaCl was more effective.

WO 01/77687 compares the removal of PrP^{C} prion proteins from a partially purified soluble preparation using specific hexapeptide ligands attached to sepharose with the removal achieved by the same sepharose material alone as reference material. SP-Sepharose and DEAE-Sepharose alone demonstrate a binding to PrP^{C} that is 100 times lower than that achieved with the hexapeptide ligand-bound resins. As a matter of fact, the document states in this respect:
“At pH 7.4 DEAE sepharose also does not appear to bind PrP^{C}.”

The low binding of SP Sepharose to PrP^{C} is still more than 20 fold reduced over the binding of PrP^{C} to silica, i.e. to an unspecific binder. From the fact that DEAE sepharose does not bind at all and that SP sepharose binds with very low and unspecific affinity to PrP^{C}, it is clear that it is the SP (sulfopropyl group) part of the SP sepharose that is responsible for the low binding affinity. Hence, WO 01/77687 actually teaches the use of sepharose as an inert solid support for PrP^{C}-specific ligands and that the SP part of SP sepharose can actually bind PrP^{C} with an affinity more than 20 fold less than that of the unspecific binder silica.

The document of P. R. Foster (Transfusion Medicine, 1999, 9, 3-14) was published in 1999, a time when prion research was still in its beginning and the scientific community had no clue regarding the physicochemical composition of prion PrP^{Sc} proteins and the detection of the causative “agent” of transmissible spongiform encephalopathy (TSE) still relied on elaborate and error prone animal studies with little quantitative significance. Furthermore, the document notes that PrP^{Sc} will generally tend to precipitate into the solids phase in a precipitation process due to its “very low aqueous solubility”. In addition, it states that PrP^{Sc} has strong hydrophilic and hydrophobic domains that will adhere to many diverse surfaces and, in particular, will interact with chromatographic and filtration media used for the production of plasma products. The document informs that ionic, cationic, hydrophobic and a number of not identified resins will bind PrP^{Sc}. Even a cellulose-acetate membrane for filtration specifically pretreated to prevent adsorption will interact with PrP^{Sc}. However, all studies presented in this document were based on a reduction of TSE infectivity and did not demonstrate any actual binding of PrP^{Sc} to any adsorbents. It is specifically noted that next to adsorbent binding a reduced PrP^{Sc} activity can also result from other mechanisms, e.g. (i) precipitation of PrP^{Sc} in solution and mechanical retention by solids such as filters and chromatographic support materials and (ii) inactivation of PrP^{Sc} by contact to solids and/or with time. In this respect the author noted in his discussion of chromatographic materials that all examined adsorbents resulted in separation of PrP^{Sc}—
“ . . . despite the use of different ligands, matrices and principles of adsorption.”

Table 1 of this document also discloses a weak reduction in PrP^{Sc} infectivity for anionic, cationic and hydrophobic ligated sepharoses when compared to other adsorbents. However, the document does not disclose any material or method for practicing its teaching relating to sepharose itself nor does it refer to any other publicly available reference for these sepharose-related embodiments. Hence, the results relating to sepharose-based adsorbents lack an enabling disclosure. Furthermore, the results of table 1 are contradicted by the specification of this document where it was demonstrated that the employed SP sepharose has a high binding affinity while Q sepharose has essentially no binding affinity to PrP^{Sc} (Table on page 28). Regarding the fidelity of the results the author notes himself:
“Much remains to be learned concerning the physicochemical properties of TSE agents in general ( . . . ) and nvCJD in particular. In the absence of such data it is inevitable that uncertainty will exist over the ability of particular process steps, either individually or in combination, to fully remove any nvCJD agent which may be present.” (emphasis added)

In other words, the author P. R. Foster himself recognized that in 1999 there were many inherent problems associated with the investigation of the potential of plasma fractionation steps to effectively reduce PrP^{Sc} and that the results of this document must be viewed as speculative and preliminary in said context.

A particularly elegant, sensitive and highly selective method for purifying and/or detecting human or animal prion proteins is based on the reversible aggregation and dissociation of prion proteins or derivatives thereof with one or more prion repeat structures that oligomerize with prion proteins at a pH of 6.2 to 7.8 and dissociate again at a pH of 4.5 to 5.5. For example, proteins with prion repeat structure(s) attached to solid support can oligomerize with prion proteins and thereby detect or remove these (PCT/EP2004 003 060).

At present, there is still a need in the art for further simple methods that concentrate, purify and/or remove prion PrP^{Sc} proteins in a simple, cost effective, highly selective and effective manner.

Due to the same amino acid sequence of PrP^{Sc} and PrP^{C} proteins both are typically concentrated and enriched together and then separated by proteinase K digestion at a later stage wherein only PrP^{C} proteins are selectively digested while PrP^{Sc} proteins remain proteinase resistant.

Hence, there is also a need for efficiently separating PrP^{Sc} from PrP^{C} proteins other than by selective enzyme digestion.

Therefore, the object underlying the present invention is the provision of a simple, low cost, efficient and highly selective method for concentrating, purifying and/or removing PrP^{Sc}.

Another object is the provision of a simple, low cost, efficient and highly selective method for separating PrP^{Sc} from PrP^{C} proteins.

The object underlying the present invention is solved by a method for concentrating and/or purifying prion PrP^{Sc} proteins and/or functional derivatives thereof, comprising the following steps:
a) contacting prion PrP^{Sc} proteins and/or functional derivatives thereof with sepharose under conditions that allow for the specific and high affinity binding of said sepharose to the prion PrP^{Sc} proteins and/or functional derivatives thereof,
b) removing the unbound non-prion proteins from the sepharose,
   wherein the sepharose is preferably not a Cu²⁺-chelating sepharose.

It was surprisingly found that sepharose by itself (i.e. as such, naked, with inactivated, removed, masked ligands) has a specific and high binding affinity to PrP^{Sc} proteins and/or functional derivatives thereof. Therefore, the binding of sepharose to PrP^{Sc} proteins and/or functional derivatives thereof is sufficient for their concentration and/or purification. One merely has to remove the unbound non-prion proteins from said sepharose.

The term “specific and high affinity binding of sepharose to prion PrP^{Sc}” as used herein is meant to indicate that the sepharose as such (i.e. the sepharose core but not any ligands thereon) binds specifically to PrP^{Sc} but not to PrP^{C}. Preferably, specific binding of sepharose in the context of the invention means the binding of sepharose as such to PrP^{Sc} multimers but not to PrP^{C}. The term high affinity binding in this respect is meant to refer to a binding affinity relating to a dissociation constant of 10⁻⁶ to 10⁻¹² M or lower, preferably 10⁻⁸ to 10⁻¹² M or lower. The skilled person can easily determine a specific and high binding affinity of a given sepharose to prion PrP^{Sc} by routine and simple binding assays. For example, one such assay would comprise the following steps:
a) providing the sepharose to be assayed and removing, inactivating and/or masking any ligands on said sepharose core if present,
b) diluting the PrP^{Sc} used to a concentration that will avoid unspecific removal, e.g. precipitation, unspecific binding, etc.,
c) incubating the sepharose of a) and PrP^{Sc} of b) in a suitable buffer under conditions and for a time that will allow for binding to each other,
d) one or more washing step(s), preferably 3 to 10 buffer volumes incubation buffer, for washing out any unbound protein from the sepharose,
e) optionally washing with an excess, preferably a 1000 fold excess, of unspecifically binding protein, preferably BSA (bovine serum albumin), in order to remove or block any unspecific binding sites on the sepharose,
f) an elution step with a buffer comprising a chaotropic agent, preferably urea and/or guanidinium chloride and/or SDS, in order to remove sepharose-bound PrP^{Sc},
g) detecting PrP^{Sc} in the eluted buffer and, thereby demonstrating high affinity binding of the sepharose to PrP^{Sc} as such.

For determining the specificity of the assayed sepharose, the above assay is repeated except that PrP^{C} instead of PrP^{Sc} is incubated in step c) and PrP^{C} is detected in the wash solution, thereby indicating the lack of binding. Alternatively, PrP^{Sc} and PrP^{C} can be incubated simultaneously with the sepharose in step c) and a specific and high affinity sepharose will result in detecting PrP^{C} in the wash solution and PrP^{Sc} in the chaotropic elution buffer only.

A more detailed and preferred assay for determining the specificity and high affinity binding of sepharoses is presented below in example 1.

In short, the term “specific and high affinity binding of sepharose to PrP^{Sc} proteins” is meant to distinguish sepharoses and methods using these from sepharoses and said methods that merely bind PrP^{Sc} unspecifically and with low affinity, e.g. by precipitation and/or low adsorption.

The terms “concentrating and/or purifying” as used herein are meant to indicate that the concentration of PrP^{Sc} proteins and/or functional derivatives thereof is raised and/or non-PrP^{Sc} proteins and/or non-protein material(s) are removed.

This method can also be employed for effectively removing prion PrP^{Sc} proteins and/or functional derivatives thereof from body fluids. In that case it comprises the following steps:
a) contacting a body fluid comprising prion PrP^{Sc} proteins and/or functional derivatives thereof with sepharose under conditions that allow for the specific and high affinity binding of the sepharose to the prion PrP^{Sc} proteins and/or functional derivatives thereof,
b) removing the body fluid from the sepharose.

Preferably said body fluid is selected from whole blood, blood fractions or brain homogenate, preferably from blood plasma. However, the body fluid may also encompass homogenates of mammalian tissues, in particular homogenates of brain tissue and spinal cord tissue.

It was also found that sepharose itself typically has an excellent compatibility with blood and as such no or at most a negligible effect on blood coagulation is observed when it is brought into contact with blood. Most ligated, metal-ligated and/or negatively charged sepharoses have also proven to be blood compatible. This is demonstrated by the results of Table 1 below, where the influence of a number of sepharoses for use according to the invention on common physiological protein parameters is tested. It was surprisingly found that sepharoses can be brought into contact with blood or blood fractions without harming or substantially altering blood parameters. Moreover, it was surprisingly found that metal-chelated sepharoses actually have a positive effect on the stability of coagulation factor VII.

TABLE 1

| Test | Resin I | Resin II | Resin III | Resin IV | Control | Unit |
|---|---|---|---|---|---|---|
| Quick* | 100 | 102 | 83 | 93 | 87 | % |
| INR** | 1.0 | 1.0 | 1.1 | 1.0 | 1.1 | |
| aPTT*** | 53 | 40 | 38 | 36 | 35 | sec. |
| Fibrinogen | 2.2 | 2.1 | 2.2 | 2.1 | 2.1 | g/l |
| Faktor V | 92 | 92 | 72 | 83 | 83 | % |
| Faktor VII | 148 | 172 | 105 | 114 | 118 | % |
| Faktor VIII | 58 | 65 | 57 | 73 | 71 | % |
| Faktor IX | 81 | 85 | 90 | 93 | 91 | % |
| Von Willebrand | 39 | 51 | 36 | 55 | 55 | % |
| Fibrin D-Dimers | 0.1 | 0.1 | 0.1 | 0.1 | 0.1 | mg/ml |
| Antithrombin | 97 | 102 | 97 | 100 | 100 | % |
| Protein C | 126 | 123 | 116 | 117 | 115 | % |
| Protein S | 167 | 119 | 146 | 149 | 135 | % |
| Protein | 62.2 | 59.1 | 56.3 | 63.1 | 60.2 | mg/ml |
| Concentration | | | | | | |

*thromboplastin time according to Quick
**international normalized ratio (INR) of thromboplastin time
***activated partial thromboplastin time (aPTT))
Resin I = Ni-Sepharose High Performance (Amersham/General Electrics 17-5268 02)
Resin II = Resin I loaded with Zn
Resin III = SP Sepharose (Sigma S 6532)
Resin IV = Sepaharose 4B (Sigma 4B-200)

Therefore, an independent aspect of the present invention is directed to novel compositions comprising coagulation factor VII, preferably the human coagulation factor VII, and further at least one metal-chelated sepharose, preferably Ni- and/or Zn-sepharose, more preferably Zn-sepharose. Another independent aspect is directed to the use of metal-chelated sepharose, preferably Ni- and/or Zn-sepharose, more preferably Zn-sepharose, for stabilizing blood, blood fractions and solid or liquid compositions comprising coagulation factor VII.

It is assumed without wishing to be bound by theory, that the advantageous effect of metal-chelated sepharoses on coagulation factor VII is based on stabilizing effects with regard to protease digestion and/or folding stability.

It should be noted that, in principle, any ligated or non-ligated sepharose can be employed for practicing the present invention(s) as long as the sepharose is not masked and, in the case that the blood is brought into contact with living cells in vivo and/or in vitro, is non-toxic. For practicing the method of the present invention for the removal of prion proteins from blood, metal-ligated sepharoses are preferred, negatively charged sepharoses are more preferred while non-ligated sepharoses and non-charged sepharoses are most preferred.

Surprisingly, the sepharose for use in the method of the present invention is not limited to any particular type of sepharose except that the sepharose core should be sufficiently accessible to the prion PrP^{Sc} proteins and/or functional derivatives thereof for binding.

Preferably, the sepharose for practicing the method of the present invention is selected from non-ligated sepharoses, more preferably selected from the group consisting of Sepharose® 2B, 4B, 6B, Sepharose® CL-4B, Sepharose®-6B, Superdex® 75, Sephacryl® 100HR and Sephadex® G10.

Also preferred for practicing the methods of the present invention are sepharoses selected from ligand-modified sepharoses, preferably selected from the group consisting of metal-chelating sepharoses, lectin agaroses, iminodiacetic sepharose, protein A agarose, streptavidin sepharose, sulfopropyl sepharose and carboxmethyl sepharose, more preferably selected from metal-chelating sepharoses and most preferred the sepharose for practicing the methods, compositions or uses is Zn-sepharose.

Zn sepharose is highly compatible with physiological fluids. Neither the sepharose nor the Zn ion will have any detrimental effects on body fluids such as whole blood, blood fractions, preferably blood plasma. Therefore, Zn sepharose is particularly useful for removing PrP^{Sc} proteins and/or functional derivatives from body fluids and/or body organs, e.g. organs for transplantation, that are to be reintroduced into an animal, preferably a human.

As mentioned before, for practicing the methods, compositions or uses of the present invention it is necessary that the optional ligands do not mask the sepharose core so that prion PrP^{Sc} proteins and/or functional derivatives thereof have free access. This is the problem with many ligand-modified sepharoses employed in the prior art. The skilled person can routinely select ligand-modified sepharoses that are sufficiently accessible for PrP^{Sc} binding by simply testing the sepharose binding affinity to PrP^{Sc} proteins, and, if desired, design appropriate ligand-modified sepharoses, e.g. by employing spacer molecules that position the ligand at an appropriate distance for the sepharose not to be masked by the ligand.

Another unexpected advantage of the method of the present invention is that the sepharose binding to prion PrP^{Sc} proteins and/or functional derivatives thereof is highly selective with respect to prion PrP^{C} proteins and/or functional derivatives thereof which do not have any significant binding affinity to sepharose by themselves.

Therefore, the method of the present invention does not only allow for selectively concentrating, purifying and/or removing prion PrP^{Sc} proteins and/or functional derivatives thereof, but actually removes the highly analogous prion PrP^{C} proteins and/or functional derivatives thereof, too.

When ligand-modified sepharoses are used, wherein the ligand part binds to prion PrP^{C} proteins and/or functional derivatives thereof, the method of the present invention allows for the simultaneous concentrating and/or purification of prion PrP^{Sc} and PrP^{C} proteins and/or functional derivatives thereof. The prion PrP^{Sc} and PrP^{C} proteins and/or functional derivatives thereof can then be separated by selectively removing PrP^{C} proteins and/or functional derivatives thereof from the sepharose.

In a preferred embodiment the present invention also relates to a method for separating and/or enriching prion PrP^{Sc} proteins and/or functional derivatives thereof from PrP^{C} proteins and/or functional derivatives thereof, comprising the following steps:
a) contacting prion PrP^{Sc} proteins and PrP^{C} proteins and/or functional derivatives thereof with ligand-modified sepharose under conditions that allow for
   (i) the specific and high affinity binding of said sepharose part to said prion PrP^{Sc} proteins and/or functional derivatives thereof, and
   (ii) the binding of said ligand part of the sepharose to PrP^{C} proteins and/or functional derivatives thereof,
b) optionally removing unbound material from said ligand-modified sepharose,
c) optionally waiting for a sufficient time period for some or most of the ligand-bound PrP^{C} proteins and/or functional derivatives thereof to convert into prion PrP^{Sc} proteins and/or functional derivatives in the close proximity of the prion PrP^{Sc} proteins and/or functional derivatives thereof,
d) adding a selective release agent to the sepharose-bound proteins and/or functional derivatives thereof from step a), b) or c) under conditions that allow for the release of PrP^{C} proteins and optionally non-prion proteins from the ligand part of the sepharose but not for the release of the prion PrP^{Sc} proteins and/or functional derivatives thereof from the sepharose part, and
e) removing the PrP^{C} and optionally non-prion proteins from the sepharose.

When prion PrP^{Sc} and PrP^{C} proteins and/or functional derivatives thereof were present on the ligand-modified sepharose it was unexpectedly found that the amount of PrP^{Sc} is raised in many instances at the expense of PrP^{C}. It is believed that PrP^{C} and/or functional derivatives thereof are converted by a spontaneous conformational change in the close proximity of PrP^{Sc} that seem to chaperone this change. This finding is in line with the understanding that the presence of PrP^{Sc} is required for PrP^{Sc} “production” from PrP^{C} precursors.

In a more preferred embodiment of the above method, said method further comprises the step of:
f) releasing PrP^{Sc} prion proteins and/or derivatives thereof from the sepharose.

For releasing PrP^{Sc} prion proteins and/or derivatives thereof from the sepharose it is preferred to add chaotropic agents and/or detergents, preferably urea and/or guanidinium chloride and/or SDS, more preferred to add urea and/or SDS, most preferred to add a gel-loading buffer comprising 8 M urea and 5% SDS and applying an electrical field. Of course, any other non-destructive method routinely applied for interrupting enzymes' affinity to polymers, preferably sugar-derived polymers, can also be employed.

For practicing the methods according to the present invention, in particular a method for separating and/or enriching prion PrP^{Sc} proteins from PrP^{C} proteins, it is preferred to employ a ligand-modified sepharose that is a metal-chelating sepharose comprising divalent immobilized metal ions.

Metal-chelating sepharoses as well as negatively charged sepharoses such as sulfopropyl sepharose and carboxymethyl sepharose may bind to PrP^{Sc} as well as PrP^{C} proteins and/or functional derivatives thereof due to the binding of the sepharose part and optionally the negative charged and/or metal ligand part of the sepharose to PrP^{Sc} and the negatively charged and/or metal ligand part of the sepharose to PrP^{C}.

The mechanism underlying the separation method of the present invention relies on the different binding properties of PrP^{Sc} and PrP^{C} regarding sepharose-immobilized metal ions. While PrP^{Sc} seems to have an intrinsic affinity to sepharose, divalent metal ions and negative charges, PrP^{C} seems to have an intrinsic affinity to divalent metal ions and negative charges only. Hence, their different affinity for sepharose can be employed for separating them.

Preferably, the metal ions of the metal-chelating sepharose are selected from the group consisting Ni²⁺, Zn²⁺, Co²⁺, Mg²⁺, Ca²⁺ and Mn²⁺.

The binding of Ca²⁺ and Mn²⁺ is weaker and both ions bind only monomers of PrP^{Sc} and PrP^{C}.

The other mentioned metal ions Ni²⁺, Co²⁺, Zn²⁺ and Mn²⁺ bind stronger to monomers and oligomers of PrP^{Sc} and PrP^{C} and are preferred for that reason. Because of its excellent binding properties and due to its lack of toxicity under physiological conditions in vivo Zn²⁺ is most preferred for the metal-chelating sepharose for practicing the methods, uses and compositions of the present invention.

Incidentally, Cu-sepharose will not retain PrP^{Sc} proteins efficiently as demonstrated in example 1. In example 1 the reloading of Ni-High Performance Sepharose with Cu²⁺ results in unspecific binding of large amounts of BSA (see also FIG. 4, lane 1) and is, therefore, not suited for the enrichment of prion proteins in complex protein solutions. Therefore, the Cu-sepharose IMAC presented by Grathwohl et al. will not provide the differential affinity necessary for a quantitative separation of PrP^{Sc} from PrP^{C}. It is therefore generally preferred for all methods of the invention that the sepharose is not a Cu²⁺-metal-chelating sepharose

When a metal-chelating sepharose is employed for practicing a method of the present invention the selective release agent is preferably a metal chelating agent, preferably an agent selected from EDTA, imidazole and/or EGTA, more preferably EDTA.

For separating PrP^{Sc} and PrP^{C} proteins and/or functional derivatives thereof from a metal chelating sepharose in a method of the invention it is most preferred that the metal is Zn²⁺ and the metal chelating agent is EDTA.

It is also preferred that the conditions in step d) of the method of the present invention for separating PrP^{Sc} and PrP^{C} proteins that allow for the release of PrP^{C} and optionally non-prion proteins from the sepharose-immobilized metal ions comprise the presence of a metal chelating agent in a concentration of 5 to 50 mM, more preferably 10 to 25 mM, most preferably EDTA at a concentration of 10 to 25 mM.

It was also found that the addition of small amounts of chelators such as EDTA, imidazole and/or EGTA to complex protein fractions such as blood fractions or brain homogenates can assist to avoid unspecific binding and therefore assists separation of unspecific material from PrP^{Sc} and/or PrP^{C} proteins. For example, for some plasma fractions it was found that 10 to 25 mM EDTA reduced unspecific binding effectively. When working with sepharose-immobilized metal ions one must take care that the effects of reducing unspecific binding and releasing PrP^{C} by chelators do not overlap if the release of PrP^{C} is not yet desired. Moreover, depending on the presence and amounts of unspecifcally binding proteins the above preferred concentration ranges will have to be adapted, i.e. increased, to compensate for the presence of unspecific proteins that scavenge the chelators for PrP^{C} release. Such an optimization is within the routine skill of those in the art.

Although sepharose itself is sufficient to bind significant amounts of PrP^{Sc} by itself if unmasked it may be desirable to employ sepharoses with at least one additional ligand for specifically binding prion PrP^{Sc} and/or PrP^{C} proteins, wherein said ligand is bound directly or indirectly, e.g. by means of a spacer molecule, to the sepharose.

In a preferred embodiment the additional ligand is selected from the group consisting of prion proteins, functional derivatives of prion proteins, His-tagged prion proteins, prion protein-binding proteins, prion protein-binding antibodies, and prion-protein specific ligands.

More preferably, the additional ligand is a prion protein, e.g. a prion fragment such as e.g. bovine PrP(25-241), that is directly or indirectly bound, e.g. by a metal chelator, to the sepharose.

As mentioned before in the introductory section, the reversible aggregation of prion proteins or derivatives thereof with one or more prion repeat structures that oligomerize with prion proteins at a pH of 6.2 to 7.8 and which may dissociate again at a pH of 4.5 to 5.5 provides highly selective and efficient means for binding, concentrating, purifying and/or removing prion proteins and/or functional derivatives thereof (PCT/EP2004 003 060). For practicing the present invention prion repeat structure(s) may be attached to sepharoses as additional ligands in order to specifically oligomerize with prion proteins and thereby to bind these.

In a more preferred embodiment the additional ligand is a prion protein and/or a functional derivative thereof.

The additional ligand on sepharoses for practicing the method of the present invention may be bound to the sepharose directly or indirectly, and is preferably bound by a spacer moiety in between the sepharose and the ligand itself.

Although the methods of the present invention are not limited to any particular prion proteins or derivatives thereof the prion proteins and/or functional derivatives thereof are selected from the group consisting of prion proteins from human, bovine, ovine, mouse, hamster, deer, or rat origin and derivatives thereof.

The term “functional derivatives of prion proteins” as used throughout the description and the claims refers to any derivatives of prion proteins, in particular fragments thereof, that comprise at least one or more prion repeat structure(s), preferably 2 to 4, more preferably 4 prion repeat structures.

In a preferred embodiment the functional derivative of a prion protein has at least one prion repeat structure(s) that is (are) an octapeptide, pseudooctapeptide, hexapeptide or pseudohexapeptide, more preferably an octapeptide having a sequence selected from the group consisting of PHGGGWGQ (SEQ ID: 1, human), PHGGSWGQ (SEQ ID: 2, mouse) and PHGGGWSQ (SEQ ID: 3, rat), or a pseudooctapeptide derived from said sequences, preferably selected from the group consisting of PHGGGGWSQ (SEQ ID: 4, various species), and PHGGGSNWGQ (SEQ ID: 5, marsupial), or a hexapeptide having a sequence selected from the group consisting of PHNPGY (SEQ ID: 6, chicken), PHNPSY (SEQ ID: 7, turtle), PHNPGY (SEQ ID: 8, turtle) or is a pseudohexapeptide derived from said sequences.

In a more preferred embodiment at least one, preferably each, of the prion repeat structures comprises an N-terminal loop conformation connected to a C-terminal β-turn structure.

Most preferred, the functional derivatives for practicing the present invention are also capable of reversible aggregation and/or dissociation, i.e. oligomerisation at a pH of 6.2 to 7.8 and/or dissociation of the oligomer aggregate at a pH of 4.5 to 5.5 in an aqueous fluid environment.

The functional derivatives of prion proteins useful for practicing the methods of the present invention may also be characterized in that they bind to unmasked sepharose to a significant extent. A significant extent means that preferably at least 50, more preferably at least 70, even more preferably at least 80, and most preferably at least 90% of the derivatives bind to unmasked sepharose relative to the naturally occurring prion protein from which the derivative is derived. For determining the extent of sepharose binding to prion protein derivatives the sepharose binding may be assessed using, e.g. Sepharose® 4 B (Sigma, product code 4B-200). The parameters for such an assay can be routinely determined by those skilled in the art.

As one of average skill in the art of prion proteins will appreciate, the functional derivatives of prion proteins mentioned herein can be briefly and sufficiently characterized in that they comprise at least one of the above prion repeat structures and are capable of binding unmasked sepharose. For bovine prion proteins or derivatives thereof, the binding of a prion protein to sepharose is assumed to be effected by domain 102-241, corresponding to amino acid residues 90 to 230 in human PrP. Analogous regions in prion proteins and derivatives thereof of other species have similar sepharose binding activity.

In a preferred embodiment the functional derivative for practicing the present invention is derived from prion proteins by one or more deletion(s), substitution(s) and/or insertion(s) of amino acid(s) and/or covalent modification(s) of one or more amino acid(s).

In a more preferred embodiment the functional derivative for practicing the present invention comprises one or more octapeptide repeat sequences, preferably amino acids 51-90, and/or the C-terminal domain, preferably, amino acids 121-230 of human PrP.

The conditions for contacting the prion PrP^{Sc} proteins and/or functional derivatives thereof with sepharose under conditions that allow for the binding of said sepharose to said prion PrP^{Sc} proteins and/or functional derivatives thereof, and optionally the binding of the ligand part of the ligand-modified sepharose to PrP^{C} proteins, if ligand-modified sepharose is employed, are preferably physiological conditions, more preferably a pH of 5 to 8 and 2 to 39° C., more preferably a pH of about 7 and about 20 to 25° C.

Further conditions for binding sepharose to prion proteins and functional derivatives thereof are ionic strength, buffer substances, etc. The person skilled in the art can routinely determine the suitable and optimized conditions for binding sepharose to prion proteins.

The term removing as it is used in the context of the removal of unbound non-prion proteins, body fluid and/or PrP^{C} proteins and/or derivatives thereof refers to standard techniques for separating proteins and sepharose material such as centrifugation, filtration, ultrafiltration, etc.

If sepharoses with the above-mentioned additional ligands for binding prion proteins by prion protein aggregation are used, naturally, a pH of 6.2 to 7.8 is preferred.

In another preferred embodiment the conditions for contacting sepharose and prion proteins comprise the presence of at least one detergent and/or a cell lysis buffer. That way, cells and/or membrane fractions present in a sample of interest can be treated by a method according to the present invention directly without any prerequisite steps for liberating the prion proteins or functional derivatives thereof and making them accessible.

In a further aspect the present invention relates to the use of sepharose, preferably ligand-modified sepharose, for concentrating, purifying and/or removing prion PrP^{Sc} proteins and/or functional derivatives thereof from other proteins in a method according to the invention.

In a preferred embodiment the sepharose is used in one of the above methods for concentrating, purifying and/or removing prion PrP^{Sc} proteins and/or functional derivatives thereof from whole blood, a blood fraction or brain homogenate, preferably from blood plasma.

In a further preferred embodiment the sepharose used is a metal-chelating sepharose, preferably comprising a divalent metal ion, more preferably a metal ion selected from the group consisting of Ni²⁺, Co²⁺, Zn²⁺ and Mn²⁺, most preferably Zn²⁺.

### FIGURES

FIG. 1 illustrates the specific binding of recombinant PrP-beta and PrP-pure to Ni Sepharose High Performance (Examples 1 and 4).

1 80 mM EDTA, 2 60 mM EDTA, 3 50 mM EDTA, 4 40 mM EDTA, 5 30 mM EDTA, 6 20 mM EDTA, 7 10 mM EDTA, 8 5 mM EDTA, 9 no EDTA, 10 standard proteins. (a) BSA (b) bovine PrP(25-241) beta form and pure form oligomers (c) bovine PrP(25-241) pure form (d) bovine PrP(25-241) beta form (e) mouse PrP(89-231) beta form.

FIG. 2 shows the binding of PrP-beta and PrP-pure to various Sepharoses (Example 1).

1 Blue Sepharose® CL-6B, 2 Iminodiacetic acid Sepharose®, 3α-Lactose-Agarose, 4 Lectin-Agarose, 5 ProteinA Sepharose®, 6 Phenyl-Sepharose® CL-6B, 7 Sepharose® CL-4B, 8 Ni Sepharose High Performance in the presence of 50 mM EDTA, 9 Ni Sepharose High Performance, 10 standard proteins. (a) BSA (b) bovine PrP(25-241) beta form and pure form oligomers (c) bovine PrP(25-241) pure form (d) bovine PrP(25-241) beta form (e) mouse PrP(89-231) beta form.

FIG. 3 depicts the binding of PrP-beta and PrP-pure to various Sepharoses (Example 1).

1 SP Sepharose®, 2 CM Sepharose®, 3 Streptavidin-Iron Oxide Particles, 4 EZview™ Red Streptavidin Affinity Gel, 5 Reactive Red 120-Agarose, 6 Iminodiacetic acid Sepharose®, 7 Sepharose® 4B, 8 Ni Sepharose High Performance in the presence of 50 mM EDTA, 9 Ni Sepharose High Performance. (a) BSA (b) bovine PrP(25-241) beta form and pure form oligomers (c) bovine PrP(25-241) pure form (d) bovine PrP(25-241) beta form (e) mouse PrP(89-231) beta form.

FIG. 4 demonstrates the binding of PrP-beta and PrP-pure to Ni Sepharose High Performance after reloading with various cations (Example 1).

1 Cu²⁺, 2 empty lane, 3 Ag⁺, 4 Mn²⁺, 5 Zn²⁺, 6 Co²⁺, 7 Ni²⁺, 8 Ni²⁺ and binding in the presence of 0.5% Triton X-100, 9 Ni²⁺ and binding in the presence of 50 mM EDTA, 10 untreated matrix. (a) BSA (b) bovine PrP(25-241) beta form and pure form oligomers (c) bovine PrP(25-241) pure form (d) bovine PrP(25-241) beta form (e) mouse PrP(89-231) beta form.

FIG. 5 illustrates the binding of PrP-beta and PrP-pure to Ni Sepharose High Performance reloaded with various cations (Example 1).

1 untreated matrix, 2 Ni²⁺ and binding in the presence of 50 mM EDTA, 3 Ni²⁺, 4 Mn²⁺, 5 Mg²⁺, 6 Ca²⁺, 7 Ni Sepharose matrix pre-loaded with BSA, 8 Ni Sepharose matrix pre-loaded with BSA. (a) BSA (b) bovine PrP(25-241) beta form and pure form oligomers (c) bovine PrP(25-241) pure form (d) bovine PrP(25-241) beta form (e) mouse PrP(89-231) beta form.

FIG. 6 shows the concentration of native PrP^{C} in various fractions of cattle blood. Ni Sepharose High Performance pre-loaded with bovine PrP(25-241) pure form was used for concentration (Example 2).

1 and 2 monocytes and lymphocytes, 3 and 4 neutrophiles, 5 and 6 platelets, 7 and 8 plasma, 9 standard protein. (a) native PrP^{C} (b) bovine PrP(25-241) pure form (c) a protein having prion protein-like characteristics.

FIG. 7 depicts the proteinase K cleavage of native PrP^{C} after concentration from monocytes and lymphocytes of cattle blood. Ni Sepharose High Performance pre-loaded with bovine PrP(25-241) pure form was used for concentration (Example 2).

1 and 2 no proteinase K, 3 5 μg/ml proteinase K 4 25 μg/ml proteinase K, 5 50 μg/ml proteinase K. (a) bovine PrP(25-241) pure form oligomer (b) native PrP^{C} (c) protease-truncated PrP^{C} (d) bovine PrP(25-241) pure form.

FIG. 8 demonstrates the proteinase K cleavage of native PrP^{C} after concentration from blood plasma of cattle. Ni Sepharose High Performance pre-loaded with bovine PrP(25-241) pure form was used for concentration (Example 2).

1 and 2 no proteinase K, 3 0.5 μg/ml proteinase K 4 5 μg/ml proteinase K, 5 50 μg/ml proteinase K. (a) native PrP^{C} (b) protease-truncated PrP^{C} (c) bovine PrP(25-241) pure form.

FIG. 9 illustrates the proteinase K cleavage of native PrP^{Sc} after concentration from buffer solution spiked with native scrapie brain homogenate. Ni Sepharose High Performance pre-loaded with bovine PrP(25-241) pure form was used for concentration (Example 3).

A In 50 mM sodium phosphate buffer. B In 0.32 M sucrose, 0.1% NP40, 0.1% deoxycholat. 1 no proteinase K, 2 5 μg/ml proteinase K 3 25 μg/ml proteinase K. (a) native PrP^{Sc} oligomer (b) native PrP^{Sc} monomeric forms.

FIG. 10 shows the proteinase K cleavage of native PrP^{C} and PrP^{Sc} after concentration from platelets of cattle blood. Ni Sepharose High Performance pre-loaded with bovine PrP(25-241) pure form was used for concentration (Example 3).

A Platelets lysate without scrape brain homogenate. B After spiking of platelet lysate with native scrapie brain homogenate. 1 no proteinase K, 2 50 μg/ml proteinase K. (a) native PrP^{Sc} oligomer (b) native PrP^{C} and PrP^{Sc} monomeric forms.

FIG. 11 depicts the separation of native PrP^{Sc} from recombinant PrP-pure. Ni Sepharose High Performance pre-loaded with bovine PrP(25-241) pure form was used for concentration (Example 4).

1 No EDTA, 2 5 mM EDTA, 3 10 mM EDTA, 4 15 mM EDTA, 5 20 mM EDTA, 6 30 mM EDTA. (a) native PrP^{Sc} oligomers (b) di-glycosylated PrP^{Sc} (c) mono-glycosylated PrP^{Sc} (d) unglycosylated PrP^{Sc} (e) bovine PrP(25-241) pure form.

FIG. 12 demonstrates the proteinase K cleavage of native PrP^{C} and PrP^{Sc} after concentration from plasma of cattle blood. Ni Sepharose High Performance pre-loaded with bovine PrP(25-241) pure form was used for concentration (Example 5).

A Cattle experimentally infected with BSE prions. B Cattle without BSE infection. 1 no proteinase K, 2 25 μg/ml proteinase K, 3 50 μg/ml proteinase K. (a) native PrP^{C} and PrP^{Sc} forms (b) bovine PrP(25-241) pure form. The four arrows indicate proteinase K cleavage products of PrP^{Sc} typically observed for cattle infected with BSE prions, but not for healthy control animals.

FIG. 13 illustrates the removal of total PrP from blood plasma of cattle. Four batches of Ni Sepharose High Performance pre-loaded with bovine PrP(25-241) pure form were used for stepwise removal (Example 6). Plasma was obtained from two blood donors A and B.

1 First removal from plasma A, 2 first removal from plasma B, 3 second removal from plasma A, 4 second removal from plasma B, 5 third removal from plasma A, 6 third removal from plasma B, 7 fourth removal from plasma A, 8 fourth removal from plasma B, 9 protein standard. (a) bovine PrP(25-241) pure form oligomer (b) native PrP^{C} (c) bovine PrP(25-241) pure form.

FIG. 14: shows the removal of total PrP from human blood plasma. Four batches of High Performance pre-loaded with human PrP(23-230) pure form were used for stepwise removal (Example 6).

1 First removal, 2 second removal, 3 third removal, 4 fourth removal. (a) bovine PrP(25-241) pure form oligomer (b) di-glycosylated native PrP^{C} (c) truncated form of native PrP^{C} (d) bovine PrP(25-241) pure form.

In the following the present invention will be further illustrated by way of examples, which relate to preferred embodiments of the present invention and which are not to be construed as limiting to the scope of the present invention.

### EXAMPLES

### Example 1

### Overall High Affinity Binding of Different Sepharoses to PrP^{Sc}

### Experimental Design:

The binding affinity and specificity of prion proteins to various Sepharoses was investigated with recombinant prion proteins in the presence of a 1,000-fold excess of BSA. The recombinant prion proteins PrP-pure (alicon ag, product code P0001) and PrP-beta (alicon ag, P 0019 and P0027) were used as model substances for PrP^{C} and PrP^{Sc}, respectively. The beta-form of bovine PrP(25-241) and mouse PrP(89-231), and the pure-form of bovine PrP(25-241) can be well distinguished by SDS-PAGE because of their different electrophoretic mobilities.

For the binding experiments 5 μg of the prion protein studied and 5 mg BSA were dissolved in 1 ml binding buffer containing 50 mM sodium phosphate pH 7. Depending of the experimental design the binding buffer contained additives such as EDTA or detergents. The mixture of Sepharose matrix and binding buffer was rotated in 1.5 ml vials for 1 h at 4° C. Subsequently, the matrix was centrifuged at 500 g and washed twice with 1 ml binding buffer to remove unbound proteins. The Sepharose-bound proteins were denatured in 10 μl standard gel-loading buffer containing 5% SDS and 8 M urea, and analysed by SDS-PAGE on 12% polyacryamide gels.

Reloading of Ni Sepharose High Performance (Amersham, Product Code 17-5268 02) with a cation of choice was performed by first washing the matrix twice with binding buffer containing 50 mM EDTA to remove bound Ni²⁺. The stripped matrix was washed twice with binding buffer, and reloaded by rotating in binding buffer containing 50 mM metal ion for 10 min at 4° C. The unbound metal ions were removed after washing twice with binding buffer.

### Results:

The results are summarized in Table 1 below: where “ ” indicates no affinity of Sepharose to PrP, “+” indicates affinity to monomeric PrP forms, “++” indicates high affinity to monomeric PrP forms, and “+++” indicates high affinity to monomeric and oligomeric forms of PrP. The terms “monomeric” and “oligomeric” PrP forms refer to disulfide-linked oligomers observed under non-reducing conditions in the SDS-PAGE rather than to aggregated PrP forms without an intermolecular disulfide bond.

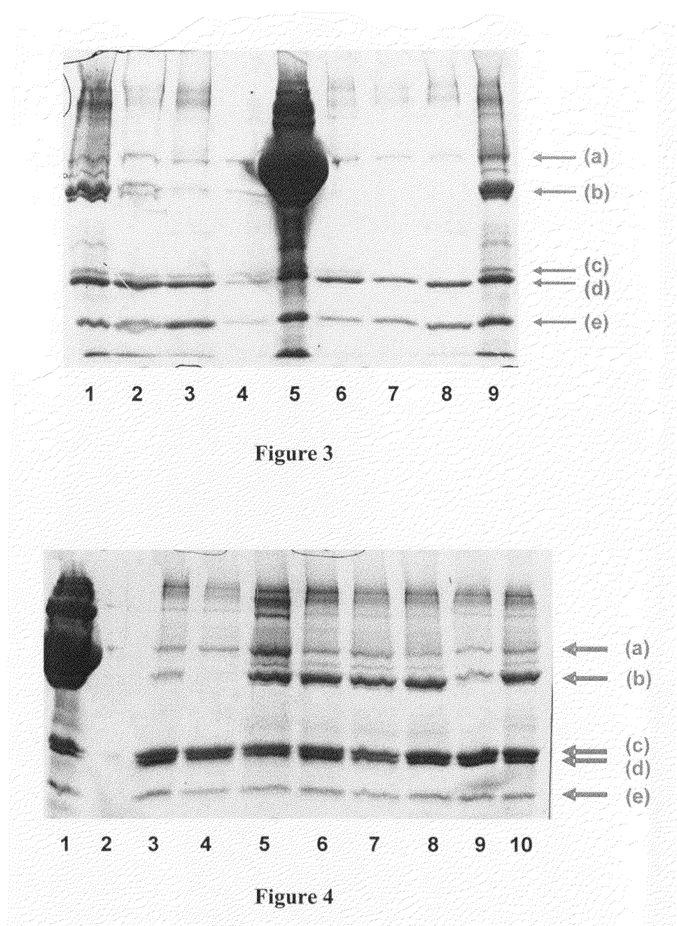

Unligated Sepharoses bind with high affinity to the beta forms of bovine PrP(25-241) and mouse PrP(89-231), but not the pure form of bovine PrP(25-241). Binding occurs to the monomeric but not the oligomeric forms (FIG. 2 lane 7; FIG. 3 lane 7). Although there is a 1000-fold excess of BSA over PrP, the relative amount of albumin bound to Sepharose matrix is relatively low, indicating that PrP binding is highly specific.

Negatively charged Sepharoses bind with high affinity to the beta form of bovine PrP(25-241) and mouse PrP(89-231), as well as the pure form of bovine PrP(25-241). Binding occurs to monomeric and oligomeric PrP forms (FIG. 3 lanes 1 and 2).

Positively charged Sepharoses showed an unspecific protein binding affinity as indicated by strong binding to BSA. Because the large amount of total protein loaded on SDS-PAGE gels, the amount of bound PrP could not be determined.

Some of the ligand-modified Sepharoses tested bind with high affinity to the beta form of bovine PrP(25-241) and mouse PrP(89-231), and the pure form of bovine PrP(25-241). Binding occurs to monomeric, but not to oligomeric PrP forms (FIG. 2 lanes 4 and 5; FIG. 3 lanes 3 and 6). However, some other ligand-modified Sepharoses showed an unspecific protein binding affinity as indicated by strong BSA binding (FIG. 2 lanes 1-2 and 6; FIG. 3 lane 5).

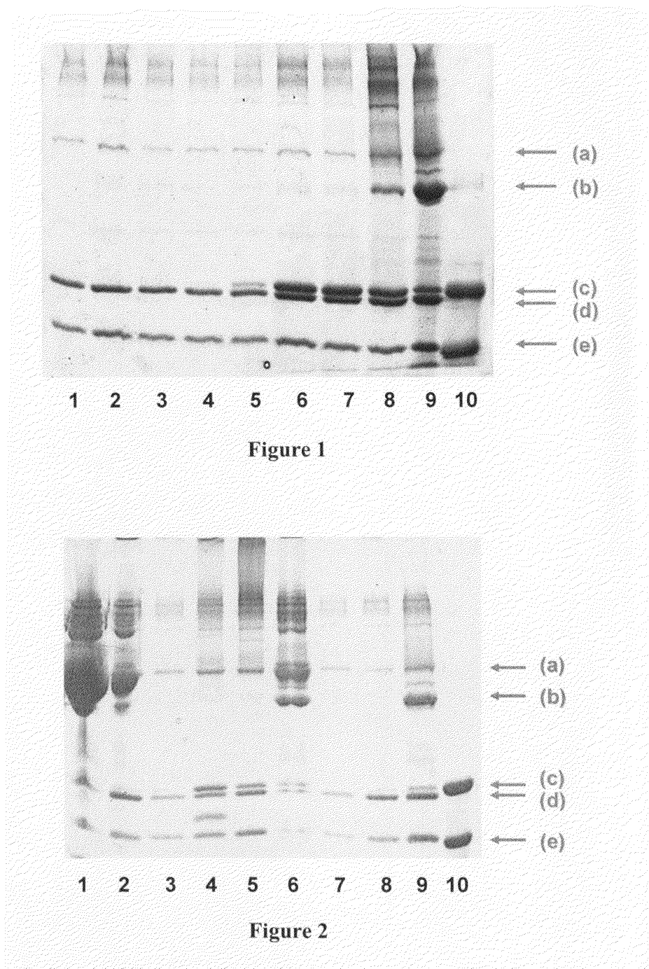

IMAC-Sepharoses bind with high affinity to the beta form of bovine PrP(25-241) and mouse PrP(89-231), as well as the pure form of bovine PrP(25-241). For some IMAC-Sepharoses, such as Ni Sepharose High Performance (Amersham), binding occurred to monomeric as well as to oligomeric PrP forms (FIG. 1 lane 9; FIG. 2 lane 9; FIG. 3 lane 9; FIG. 4 lane 10). However, many Sepharoses exclusively bound to monomeric PrP.

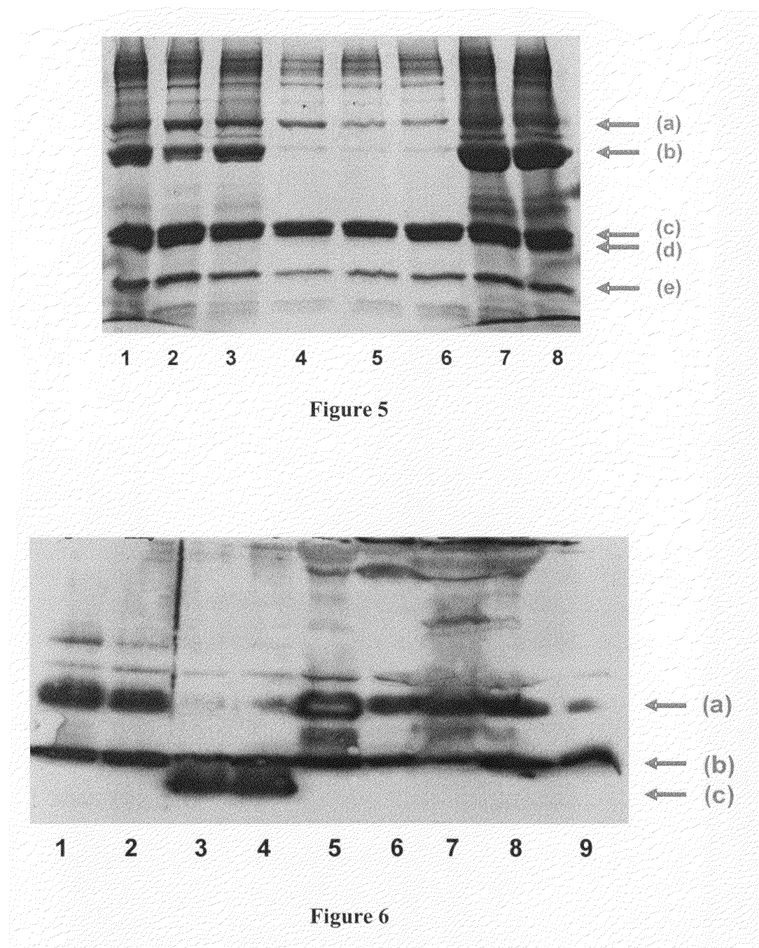

The binding of IMAC Sepharose to prion protein is modulated by the type of chelated metal ions. Ni Sepharose High Performance reloaded with Ni²⁺, Zn²⁺, or Co²⁺ binds with high affinity to the beta form of bovine PrP(25-241) and mouse PrP(89-231), as well as the PrP-pure form of bovine PrP(25-241) (FIG. 4 lanes 5, 6, 7, and 10). The binding to the oligomeric PrP forms to Ni Sepharose High Performance remains unchanged after washing with 0.5% Triton X-100 (FIG. 4 lane 8), indicating that binding is specific. Pre-coating of Ni Sepharose High Performance with BSA results in more efficient binding of oligomeric PrP-forms (FIG. 5 lanes 7-8). Reloading of Ni Sepharose High Performance with Cu²⁺ results in unspecific binding of large amounts of BSA (FIG. 4 lane 1), and is thus not applicable for specific enrichment of prion proteins in complex protein solutions. Ni Sepharose High Performance reloaded with Mn²⁺, Mg²⁺ or Ca²⁺ predominantly binds to monomeric PrP (FIG. 4 lane 4; FIG. 5 lane 4-6).

### Interpretation:

The binding of PrP-beta to Sepharoses is modulated by the:
accessibility of the Sepharose matrix
presence of Sepharose-immobilize metal ions
presence of negative charges on the Sepharose

The binding of PrP-pure to Sepharoses is modulated by the:
presence of Sepharose-immobilize metal ions
presence of negative charges on the Sepharose

The amino acids responsible for the intrinsic affinity of the beta form to Sepharose are located within residues 104 to 241 of the bovine prion protein sequence. Residues 25 to 103 containing the octapeptide repeats are thus not required for Sepharose binding. However, the presence of residues 23 to 103 results in an increased affinity to IMAC Sepharose or Cation Exchange Sepharose by binding of immobilized metal ions and negative charges, respectively.

### Summary:

Unligated Sepharose has an intrinsic binding affinity for PrP-beta (corresponding to PrP^{Sc}) but not PrP-pure (corresponding to PrP^{C}). Thus unligated Sepharoses can be used for concentrating, purifying, and removing prions without affecting the concentration of PrP^{C}.

The binding affinity of PrP-beta to Sepharose is increased when the matrix is modified with immobilized metal ions (such as Ni²⁺, Zn²⁺, Co²⁺) or negative charges (such as sulfopropyl or carboxymethyl), where these ligands also bind to PrP-pure. Thus IMAC Sepharoses and negatively charged Sepharoses can be used for concentrating, purifying, and removing of various prion protein forms.

### Example 2

### Concentration of Native Prion Proteins in Blood

### Experimental Design:

The amount of PrP^{C} in blood of healthy humans and animals is only marginal. Without any concentration step PrP^{C} is not detected using conventional analytical methods such as Western Blot. However, applying Ni Sepharose High Performance pre-loaded with bovine PrP(25-241) pure-form to 20 ml blood, PrP^{C} becomes visible.

Ni Sepharose High Performance pre-loaded with bovine PrP(25-241) was prepared by adding 5 ng of the recombinant prion protein to 20 ml of the Sepharose equilibrated with 50 mM phosphate buffer. The mixture was vortexed, and incubated while rotating for 1 h at 4° C.

The preparation of cell lysates and plasma from fresh cattle blood was carried out using standard protocols. For Example, the plasma fraction was prepared from 20 ml blood collected in EDTA tubes, after 1/10 dilution with sodium citrate to a final concentration of 10 mM. The citrate blood was diluted 1/1 with Gey's balanced salt solution (Sigma, Product Code G9779) and mixed carefully. The solution was distributed to 50 ml Falcon tubes with a maximal volume of 15 ml per tube, and centrifuged at 200 g for 7 min with brake on. To the supernatant EDTA was added to a final concentration of 10 mM, and centrifuged at 560 g for 10 min with brake on. Native blood PrP was concentrated by adding 60 μl of Ni Sepharose High Performance pre-loaded with bovine PrP(25-241) to each blood fraction. The protein solutions were incubated while rotating for 1 h at 4° C., and centrifuged at 500 g for 2 min. The supernatant was discarded, and the Sepharose was washed twice with 1 ml buffer containing 100 mM sodium phosphate, 10 mM Tris, 20 mM imidazole, pH 8 to remove unbound proteins. For consecutive proteinase K digest each blood fraction was divided into three parts. The Sepharose-bound proteins were incubated with proteinase K (Sigma, P2308) at concentrations between 0 μg/ml and 50 μg/ml, while shaking in an Eppendorf Thermomixer at 1400 rpm for 1 h at 37° C. The sample volume was 80 μl in 0.2 ml PCR tubes, and the cleavage buffer was composed of 50 mM sodium phosphate pH 7 and 150 mM NaCl. To guarantee a homogeneous distribution of the Sepharose matrix during proteinase K reaction, 10 μl-tips (Treff) cut to a length of 0.5 cm were added to the PCR tubes. The reaction was stopped by adding 2 μl of a 150 mM PMSF stock solution. The tubes were vortexed and centrifuged at 500 g for 2 min, and the supernatant was discarded. The Sepharose-bound protein was denatured in 10 μl gel-loading buffer containing 5% SDS and 8 M urea, and loaded onto a 12% acrylamide gel. Proteins were transferred to PVDF using a semi-dry discontinuous three-buffer system. Transfer was at 1 mA/cm² for 1 h. Blots were analysed using the standard protocol of ECL Advance Western Blotting Detection Kit (Amersham), a PrP-specific monoclonal antibody, and a peroxidase-coupled anti-mouse monoclonal antibody.

### Results:

After concentration nanogram-amounts PrP^{C} are measured in various blood fractions, including monocytes and lymphocytes, platelets, and plasma (FIG. 6). Native PrP^{C} in blood cells and plasma predominantly is di-glycosylated and has an apparent molecular weight of about 35 kDa. Neutrophiles do not express significant amounts of prion protein.

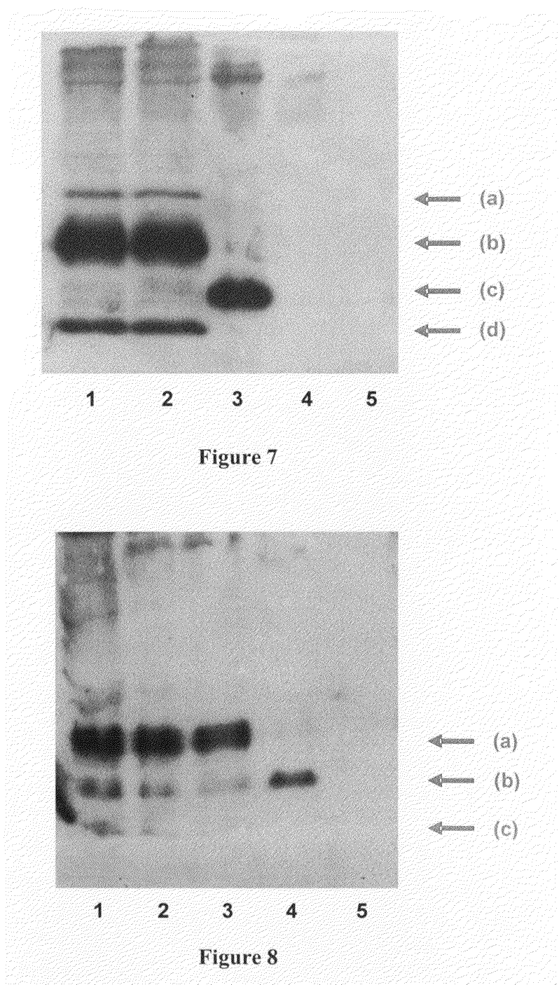

Sepharose-bound PrP is accessible to proteinase K digestion. After treatment of immobilized prion protein from cell lysates or plasma with 5 μg/ml proteinase K for one hour, PrP^{C} is partially degraded showing an apparent molecular weight of about 30 kDa (FIGS. 7 and 8). At 10-fold higher proteinase K concentration prion protein is completely degraded.

### Summary:

IMAC-Sepharose constitutes an excellent matrix for concentration of total prion protein from body fluids. Sepharose-immobilized prion proteins are accessible for further biochemical analysis employed in prion diagnostics, such as protease digestion.

### Example 3

### Concentrating PrP^{Sc} in Blood after Spiking with Brain Homogenate

### Experimental Design:

The nature of native PrP^{Sc} in blood is not known, although it seems likely that it has similar biochemical properties as PrP^{Sc} found in brain. We used PrP^{Sc} from brain homogenate (PrP^{Sc} concentration between 1 pg/ml and 1 ng/ml) as a model substrate to analyse its binding to Ni Sepharose High Performance pre-loaded with bovine PrP(25-241).

The concentration experiment was carried out as described under Example 2, except that various amounts of scrapie brain homogenate were added to the samples.

### Results:

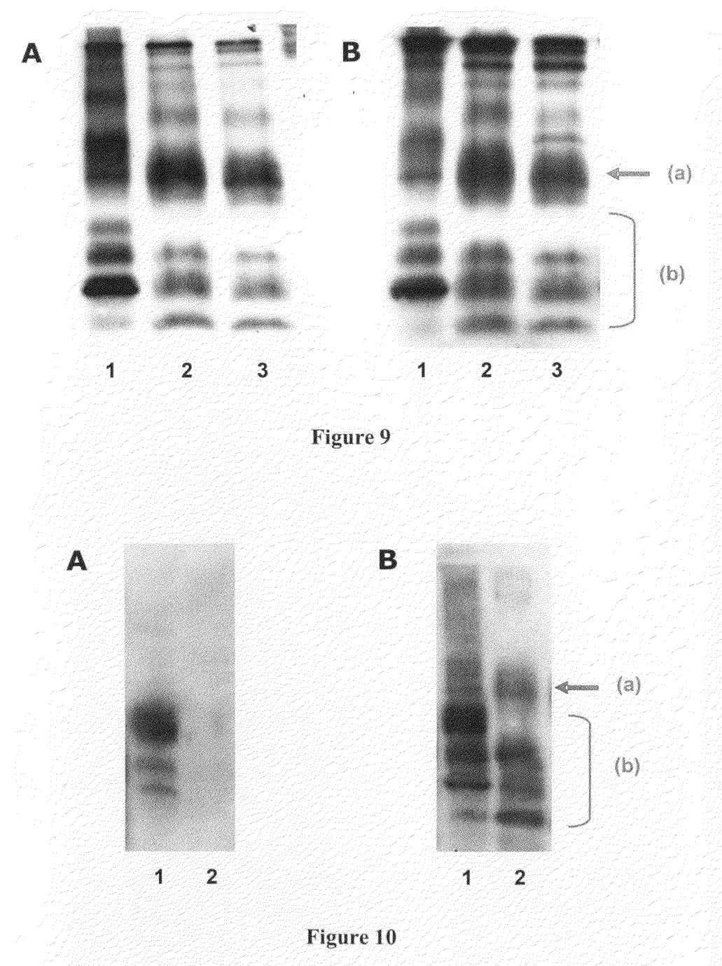

After spiking of 1 ml sodium phosphate buffer pH 8 with brain homogenate to a final concentration of 1 ng/ml PrP^{Sc} and subsequent 200-fold concentration, di-glycosylated, mono-glycosylated, and unglycosylated PrP^{Sc} as well as a multimeric forms could be detected in the Western Blot (FIG. 9). Thus, independent of its aggregation and glycosylation state, PrP^{Sc} efficiently binds to the Sepharose. In the presence of 5 and 25 μg/ml proteinase K about 70 residues are removed from the N-terminus of immobilized PrP^{Sc}. Similar results are obtained up to 5.000-fold concentration of PrP^{Sc}, and in phosphate buffer containing 0.5% Triton X-100, 0.5% deoxycholat, and 0.43% sucrose. Even after N-terminal truncation the binding of PrP^{Sc} to the Sepharose is not diminished by the presence of detergent or carbohydrate.

Similar results were obtained with platelets lysate and plasma. Native blood PrP^{C} and PrP^{Sc} from brain homogenate were co-concentrated by the Sepharose matrix. In the presence of 5 μg/ml proteinase K native PrP^{C} was completely degraded (FIG. 10 A), whereas concentrated PrP^{Sc} showed the typical pattern of di-glycosylated, mono-glycosylated, and unglycosylated forms (FIG. 10 B).

### Summary:

IMAC-Sepharose constitutes an excellent matrix for concentration of infectious prions from body fluids. Sepharose-immobilized PrP^{Sc} is accessible for further biochemical analysis employed in prion diagnostics, such as proteinase K digestion.

### Example 4

### Conformation-Specific Elution of Concentrated Prions Proteins

### Experimental Design:

As mentioned in the previous Examples, Ni Sepharose High Performance binds with high affinity to the recombinant proteins PrP-beta and PrP-pure, as well as to native PrP^{C} and PrP^{Sc}.

To investigate the elution properties of the Sepharose matrix, we used the same experimental design as before, with the sole exception that the binding buffer contained various concentrations of EDTA.

### Results:

In the presence of 10 mM EDTA, exclusively the dimeric forms of recombinant PrP are released from the Sepharose matrix. In the presence of 40 mM EDTA the pure form of bovine PrP(25-241) is released, whereas the beta forms stay bound to the Sepharose even at 80 mM EDTA concentration (FIG. 1).

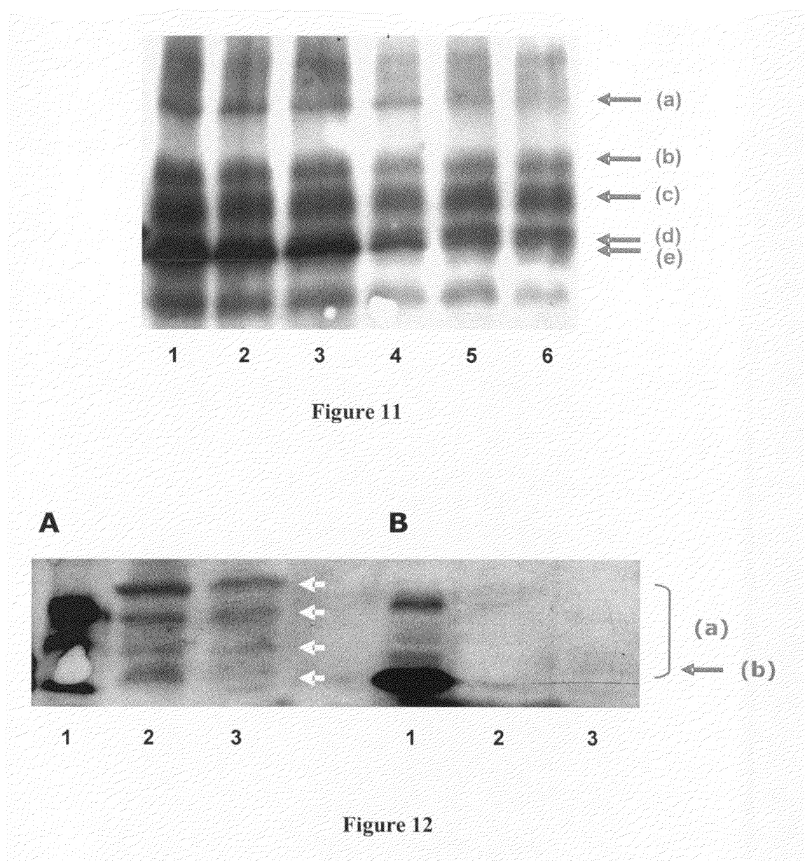

The three glycoforms of PrP^{Sc} and recombinant bovine PrP(25-241) are co-concentrated, when treated with Ni Sepharose High Performance. After washing the Sepharose matrix with increasing concentrations of EDTA the bovine PrP(25-241) is gradually released, whereas the PrP^{Sc} stays bound (FIG. 11). Thus, the pure form representing native PrP^{C} is specifically released from the Sepharose. Similar results were obtained with native PrP^{C} from blood after spiking with scrapie brain homogenate.

### Interpretation:

Addition of EDTA to Ni Sepharose High Performance results in stripping of Ni²⁺ from the Sepharose. At a concentration of EDTA where the amount Sepharose-immobilized Ni⁺ falls below a certain value, there are not enough binding sites available and PrP^{C} is released from the Sepharose. In contrast, PrP^{Sc} stays bound, because of its additional Sepharose binding activity.

### Summary:

IMAC-Sepharose constitutes an excellent matrix for concentration of PrP^{C} and PrP^{Sc} from body fluids, and subsequent separation of the two PrP conformers in the presence of EDTA.

### Example 5

### Detection of Native PrP^{Sc} in Blood from BSE-Infected Cattle

### Experimental Design:

The amount of PrP^{Sc} in blood of cattle infected with BSE prions is only marginal. Without any concentration step PrP^{Sc} is not detected using conventional analytical methods such as Western Blot. However, applying Ni Sepharose High Performance pre-loaded with bovine PrP(25-241) pure-form to 20 ml blood of a cow experimentally infected with BSE, PrP^{Sc} becomes visible.

For these experiments we use the same experimental setup as in Example 2.

Results:

After treatment of immobilized prion protein from plasma with 25 μg/ml or 50 μg/ml proteinase K, there is an accumulation of four prion protein bands that are typically detected for cattle infected with BSE (FIG. 12 A). Picogram-amounts of PrP^{Sc} shifted relative to undegraded PrP^{C} in the absence of proteinase K. No such bands are observed for control cattle. (FIG. 12 B).

### Summary:

IMAC-Sepharose constitutes an excellent matrix for the detection of native PrP^{Sc} from body fluids of BSE-infected cattle.

### Example 6

### Removal of Native Prion Proteins in Blood by Filtration

### Experimental Design:

From the previous examples it turned out that the small amounts of Sepharose matrix used have a binding capacity in the nanogram range. The Sepharose thus may be applied for complete removal of total prion proteins from body fluids such as human and animal blood plasma.

For the plasma filtration experiments we used the same experimental setup as described in Example 2, except that four batches of Sepharose matrix were added consecutively to the same plasma. The Ni Sepharose High Performance for filtration of human and cattle plasma was pre-loaded with the pure form of human PrP(23-230) bovine PrP(25-241), respectively.

### Results:

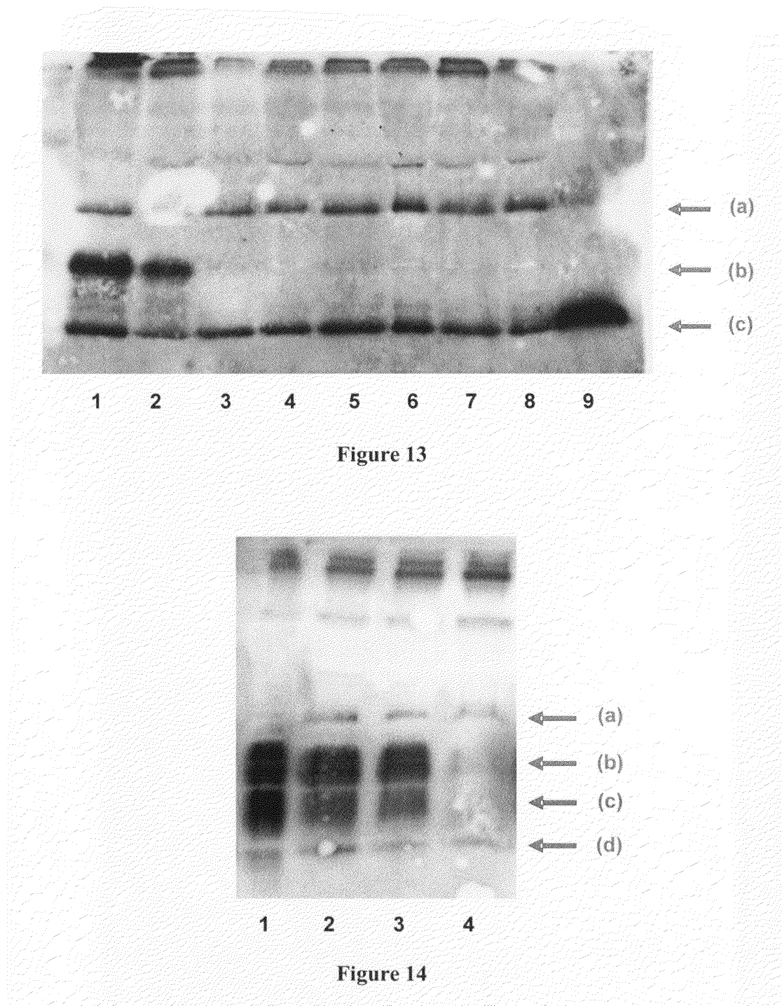

The first batch of 20 μl Ni Sepharose High Performance pre-loaded with bovine PrP(25-241) pure-form binds nanogram-amounts of native prion protein after 1 hour of incubation in 10 ml plasma from cattle blood (FIG. 13). The second batch of Sepharose already is completely free of prion protein up to the detection limit of 1 pg. The same result was obtained for the third and fourth batch of Sepharose. Thus, all prion proteins have been removed from plasma already after the first incubation period with the Sepharose matrix.

The first batch of 20 μl Ni Sepharose High Performance pre-loaded with human PrP(23-230) pure-form also binds nanogram-amounts of native prion protein after 1 hour of incubation in 10 ml human plasma (FIG. 14). The second and third batches of Sepharose bind relatively less prion protein when compared to the previous batch, respectively. The fourth batch of Sepharose is completely free of prion protein up to the detection limit of 1 pg. Thus, all prion proteins have been removed from human plasma.

The larger amount of Sepharose required for filtration of human plasma when compared to cattle plasma is explained by an about 4-fold higher amount of PrP^{C} in human plasma.

### Summary:

IMAC-Sepharose constitutes an excellent matrix for the removal of native prion proteins from body fluids such as human and bovine plasma.

TABLE 2

| | | | | Mouse | Bovine | |
|---|---|---|---|---|---|---|
| | | | | PrP | PrP | |
| | | | Bovine | (89-230) | (25-241) | |
| | | Product | PrP(25-241) | beta | pure | |
| Resin | Company | Code | beta form | form | form | BSA |
| Unligated- | | | | | | |
| Sepharoses | | | | | | |
| Sephacryl ® 100-HR | Sigma | S-100-HR | ++ | | − | − |
| Sephadex ® G10 | Sigma | G-10-120 | ++ | | − | − |
| Sepharose ® 2B | Sigma | 2B-300 | ++ | | − | − |
| Sepharose ® 4B | Sigma | 4B-200 | ++ | ++ | − | − |
| Sepharose ® 6B | Sigma | 6B-100 | + | | − | − |
| Sepharose ® CL-4B | Sigma | CL-4B-200 | ++ | ++ | − | − |
| Sepharose ® CL-6B | Sigma | CL-6B-200 | ++ | | − | − |
| Superdex ® 75 | Sigma | S 6657 | ++ | | − | − |
| Negatively | | | | | | |
| Charged | | | | | | |
| Sepharoses | | | | | | |
| SP Sepharose ® | Sigma | S 6532 | +++ | ++ | +++ | − |
| CM Sepharose ® | Sigma | CCL-6B- | ++ | ++ | + | − |
| | | 100 | | | | |
| Positively | | | | | | |
| Charged | | | | | | |
| Sepharoses | | | | | | |
| DEAE Sepharose ® | Sigma | DCL-6B- | − | − | − | +++ |
| | | 100 | | | | |
| Q Sepharose ® Fast | Sigma | Q 1126 | − | − | − | +++ |
| Flow | | | | | | |
| Ligand-Modified | | | | | | |
| Sepharoses | | | | | | |
| α-Lactose-Agarose | Sigma | L 7634 | ++ | ++ | + | − |
| Iminodiacetic acid | Sigma | I 4510 | ++ | ++ | + | − |
| Sepharose ® | | | | | | |
| Streptavidin-Iron | Sigma | S-2415 | ++ | ++ | + | − |
| Oxide Paricles | | | | | | |
| ProteinA | Sigma | P 3391 | ++ | ++ | ++ | − |
| Sepharose ® | | | | | | |
| Lectin-Agarose | Sigma | L 4018 | ++ | ++ | ++ | − |
| Blue Sepharose ® | Sigma | R 8752 | ++ | ++ | ++ | ++ |
| CL-6B | | | | | | |
| Reactive Red 120- | Sigma | R 6143 | ++ | ++ | ++ | +++ |
| Agarose | | | | | | |
| Phenyl- | Sigma | P 7892 | ++ | + | ++ | + |
| Sepharose ® CL-4B | | | | | | |
| EZview ™ Red | Sigma | E-5529 | + | + | + | − |
| Streptavidin Affinity | | | | | | |
| Gel | | | | | | |
| Heparin Sepharose | Amersham | 17-0998 | + | + | +++ | ++ |
| 6 Fast Flow | | | | | | |
| IMAC-Sepharoses | | | | | | |
| Ni Sepharose High | Amersham | 17-5268 02 | +++ | ++ | +++ | − |
| Performance | | | | | | |
| HisTrap HP | Amersham | 17-5247-01 | +++ | ++ | +++ | − |
| His-Select ™ Nickel | Sigma | P 6611 | ++ | | − | − |
| Affinity Gel | | | | | | |
| His-Select ™ Nickel | Sigma | H 1786 | ++ | ++ | ++ | − |
| Magnetic Beads | | | | | | |
| EZview ™ Red His- | Sigma | E 3528 | ++ | ++ | ++ | − |
| Select ™ Nickel | | | | | | |
| Affinity Gel | | | | | | |
| Chelating | Amersham | 17-0575-01 | ++ | ++ | ++ | − |
| Sepharose Fast | | | | | | |
| Flow | | | | | | |
| HisTrap FF | Amersham | | ++ | ++ | + | − |
| His-Select ™ HF | Sigma | H 0537 | ++ | + | + | − |
| Nickel Affnity Gel | | | | | | |
| Ni-NTA Agarose | Quiagen | 1018240 | + | | − | − |
| His-Select ™ Cobalt | Sigma | H 8162 | + | + | + | − |
| Affinity Gel | | | | | | |
| His-Select ™ Nickel | Sigma | H 8286 | + | + | + | − |
| Cartridges | | | | | | |

+++ PrP^{Sc} monomer and multimer binding
++ PrP^{Sc} monomer binding
+ PrP^{Sc} monomer binding but with lower affinity than ++
− no PrP^{Sc} binding

1. A method for concentrating and/or purifying prion PrP^{Sc} proteins and/or functional derivatives thereof, comprising the following steps:
a) contacting prion PrP^{Sc} proteins and/or functional derivatives thereof with sepharose under conditions that allow for the specific and high affinity binding of said sepharose to said prion PrP^{Sc} proteins and/or functional derivatives thereof,
b) removing the unbound non-prion proteins from said sepharose,
wherein the sepharose is not a Cu²⁺-chelating sepharose.

2. A method for removing prion PrP^{Sc} proteins and/or functional derivatives thereof from body fluids, comprising the following steps:
a) contacting a body fluid comprising prion PrP^{Sc} proteins and/or functional derivatives thereof with sepharose under conditions that allow for the specific and high affinity binding of said sepharose to said prion PrP^{Sc} proteins and/or functional derivatives thereof,
b) removing the body fluid from said sepharose.

3. The method of claim 2, wherein the body fluid is selected from whole blood, blood fractions or brain homogenate, preferably from blood plasma.

4. The method according to claim 1, wherein the sepharose is selected from unligated sepharoses, preferably selected from the group consisting of Sepharose 2B®, 4B®, 6B®, Sepharose CL-4B®, Sepharose-6B®, Superdex 75®, Sephacryl 100HR® and Sephadex G10®.

5. The method according to claim 1, wherein the sepharose is selected from ligand-modified sepharoses, preferably selected from the group consisting of metal-chelating sepharoses, lectin agaroses, iminodiacetic sepharose, protein A agarose, streptavidin sepharose, sulfopropyl sepharose and carboxmethyl sepharose, more preferably selected from metal-chelating sepharoses, most preferably the sepharose is Zn sepharose.

6. A method for separating and/or enriching prion PrP^{Sc} proteins and/or functional derivatives thereof from PrP^{C} proteins and/or functional derivatives thereof, comprising the following steps:
a) contacting prion PrP^{Sc} proteins and PrP^{C} proteins and/or functional derivatives thereof with ligand-modified sepharose under conditions that allow for
(i) the specific and high affinity binding of said sepharose part to said prion PrP^{Sc} proteins and/or functional derivatives thereof, and
(ii) the binding of said ligand part of the sepharose to PrP^{C} proteins and/or functional derivatives thereof,
b) optionally removing unbound material from said ligand-modified sepharose,
c) optionally waiting for a sufficient time period for some or most of the ligand-bound PrP^{C} proteins and/or functional derivatives thereof to convert into prion PrP^{Sc} proteins and/or functional derivatives in the close proximity of the prion PrP^{Sc} proteins and/or functional derivatives thereof,
d) adding a selective release agent to the sepharose-bound proteins and/or functional derivatives thereof from step a), b) or c) under conditions that allow for the release of PrP^{C} proteins and optionally non-prion proteins from the ligand part of the sepharose but not for the release of the prion PrP^{Sc} proteins and/or functional derivatives thereof from the sepharose part, and
e) removing the PrP^{C} and optionally non-prion proteins from the sepharose.

7. The method of claim 6, further comprising the step of:
f) releasing PrP^{Sc} prion proteins and/or derivatives thereof from the sepharose.

8. The method of claim 7, wherein the release of PrP^{Sc} prion proteins and/or derivatives thereof is accomplished by adding chaotropic agents and/or detergents, preferably urea and/or guanidinium chloride and/or SDS, more preferably adding urea and/or SDS, most preferably adding a gel-loading buffer comprising 8 M urea and 5% SDS and applying an electrical field.

9. The method of claim 5, wherein the ligand-modified sepharose is a metal-chelating sepharose comprising divalent immobilized metal ions.

10. The method of claim 9, wherein the metal ions are selected from the group consisting Ni²⁺, Co²⁺, Zn²⁺, Mg²⁺, Ca²⁺ and Mn²⁺.

11. The method of claim 10, wherein the metal ions are selected from the group consisting Ni²⁺, Co²⁺, Zn²⁺ and Mn²⁺.

12. The method of claim 11, wherein the metal ions are Zn²⁺.

13. The method of claim 6, wherein the ligand-modified sepharose is a metal-chelating sepharose according to any one of claims 9 to 12 and the selective release agent is a metal chelating agent, preferably an agent selected from EDTA, imidazole and/or EGTA.

14. The method of claim 13, wherein the metal chelating agent is EDTA.

15. The method according to claim 14, wherein the metal chelating sepharose comprises Zn²⁺ and the metal chelating agent is EDTA.

16. The method according to claim 6, wherein the conditions in step d) of claim 6 that allow for the release of non-prion proteins and PrP^{C} from the sepharose-immobilized metal ions comprise the presence of a metal chelating agent in a concentration of 5 to 50 mM, more preferably 10 to 25 mM, most preferably EDTA at a concentration of 10 to 25 mM.

17. The method of claim 1, wherein at least one additional ligand for binding prion PrP^{Sc} and/or PrP^{C} proteins is bound directly or indirectly to the sepharose.

18. The method of claim 17, wherein the additional ligand is selected from the group consisting of prion proteins, functional derivatives of prion proteins, His-tagged prion proteins, prion protein-binding proteins, prion protein-binding antibodies, and prion-protein specific ligands.

19. The method of claim 18, wherein the additional ligand is a prion protein and/or a functional derivative thereof.

20. The method of claim 17, wherein the additional ligand is bound to sepharose directly or indirectly, preferably by a spacer moiety.

21. The method according to claim 1, wherein the prion proteins and/or functional derivatives thereof are selected from the group consisting of prion proteins from human, bovine, ovine, mouse, hamster, deer, or rat origin and derivatives thereof.

22. The method of claim 1, wherein the functional derivative is derived from prion proteins by one or more deletion(s), substitution(s) and/or insertion(s) of amino acid(s) and/or covalent modification(s) of one or more amino acid(s).

23. The method of claim 1, wherein the functional derivative comprises one or more octapeptide repeat sequences, preferably amino acids 51-90, and/or the C-terminal domain, preferably, amino acids 121-230, of human PrP.

24. The method of claim 1, wherein the conditions for the binding of sepharose to prion PrP^{Sc} proteins and/or functional derivatives thereof are physiological conditions, preferably a pH of 5 to 8 and 2 to 39° C., more preferably a pH of about 7 and about 2 to 8° C.

25. The method of claim 24, wherein the conditions comprise the presence of at least one detergent and/or a cell lysis buffer.

26. Use of sepharose having specific and high affinity binding to PrP^{Sc} for concentrating, purifying and/or removing prion PrP^{Sc} proteins and/or functional derivatives thereof from other proteins in a method according to claim 1.

27. The use of sepharose according to claim 26 for concentrating, purifying and/or removing prion PrP^{Sc} proteins and/or functional derivatives thereof from whole blood, a blood fraction or brain homogenate, preferably from blood plasma.

28. The use of claim 26, wherein the sepharose is a metal-chelating sepharose, preferably comprising a divalent metal ion, more preferably a metal ion selected from the group consisting of Ni²⁺, Co²⁺, Zn²⁺ and Mn²⁺, most preferably Zn²⁺.


SEQUENCE LISTING (8 sequences)

SEQ ID NO: 1 - protein, 8 aa, Homo sapiens
PHGGGWGQ

SEQ ID NO: 2 - protein, 8 aa, Mus musculus
PHGGSWGQ

SEQ ID NO: 3 - protein, 8 aa, Rattus norvegicus
PHGGGWSQ

SEQ ID NO: 4 - protein, 9 aa, Artificial Sequence
  peptide derivative of a prion protein found in various vertebrate species
PHGGGGWSQ

SEQ ID NO: 5 - protein, 10 aa, Artificial Sequence
  peptide derivative of a prion protein from a marsupial species
PHGGGSNWGQ

SEQ ID NO: 6 - protein, 6 aa, Gallus gallus
PHNPGY

SEQ ID NO: 7 - protein, 6 aa, Artificial Sequence
  hexapeptide derivative of a prion protein
PHNPSY

SEQ ID NO: 8 - protein, 6 aa, Artificial Sequence
  hexapeptide derivative of a prion protein from a turtle species
PHNPGY